(19) United States
(12) Patent Application Publication
(10) Pub. No.: US 2006/0189931 A1
(43) Pub. Date: Aug. 24, 2006

(54) Syringe with retractable needle

(76) Inventor: John Riemelmoser, SOUTH AUSTRALIA (AU)

Correspondence Address: Klauber & Jackson;Continental Plaza, 411 Hackensack Ave., 4th Floor, Hackensack, NJ, 07601, US

(21) Appl. No.: 10/550,604

(22) PCT Filed: Mar. 24, 2003
(86) PCT No.: PCT/AU03/00346

§ 371 Date: Sep. 22, 2005

(51) Int. Cl. A61M 5/00; A61M 5/32

(52) U.S. Cl. 604/110; 604/198; 128/919

(57) ABSTRACT

A syringe retraction arrangement adapted to automatically retract a syringe (2) into a container (40) upon full depression of a plunger (20) of the syringe into the syringe thereby avoiding the potential for needle stick injuries. The syringe (2) is resiliently biased to retract into the container and has a retaining arrangement to prevent it retracting and has release means for the retaining arrangement which are engaged upon complete depression of the plunger to operate the release means. The arrangement has a sleeve assembly (41, 42) to receive the syringe with an aperture (45) at one end of the sleeve assembly through which the hypodermic needle extends in use, a pair of ears (55) on the sleeve on the end remote from the aperture. The ears are adapted to engage the end of the syringe barrel (23) remote from the hypodermic needle and to release the syringe barrel by disengagement with the end of the barrel when the ears are spread apart. A spring (37) moves the syringe barrel within the sleeve assembly to withdraw the hypodermic needle into the sleeve tube when the end of the syringe barrel is released from the ears.

### FIELD OF INVENTION

This invention relates to a syringe with a needle protection system and in particular to an arrangement that ensures that the needle of the syringe retracts into a container to ensure that it is totally encapsulated to thereby prevent needle stick injuries. The invention also relates to a single use syringe arrangement.

### BACKGROUND OF THE INVENTION

Needle stick injuries resulting from contact with discarded needles are an increasing problem. The handling of syringes by medical personnel also exposes them to accidental needle stick injuries.

It is the object of this invention to provide means whereby after use the needle of a syringe is retracted to a position where it is shielded to avoid the potential for needle stick injuries.

### BRIEF DESCRIPTION OF THE INVENTION

In one form therefore although this may not necessarily be the only or broadest form the invention is said to reside in a syringe retraction arrangement adapted to automatically retract a syringe into a container upon full depression of a plunger of the syringe into the syringe thereby avoiding the potential for needle stick injuries.

Preferably the syringe is resiliently biased to retract into the container and has a retaining arrangement to prevent it retracting and has release means for the retaining arrangement which are engaged upon complete depression of the plunger to operate the release means.

In a further form the invention is said to reside in a sleeve assembly and syringe arrangement, the syringe being of a type having a syringe barrel with a plunger bulb and a plunger arm terminating in a plunger flange, the plunger adapted to move in the syringe barrel, a pair of finger flanges on the syringe barrel and a hypodermic needle extending from the syringe barrel, the sleeve assembly including a sleeve to receive the syringe with an aperture at one end of the sleeve through which the hypodermic needle extends in use, a pair of ears on the sleeve on the end remote from the aperture, the ears adapted to engage the end of the syringe barrel remote from the hypodermic needle and to release the syringe barrel by disengagement with the end of the barrel when the ears are spread apart and means to move the syringe barrel within the sleeve assembly so as to move the syringe to withdraw the hypodermic needle into the sleeve tube when the end of the syringe barrel is released from the ears.

Preferably the end of the syringe barrel is adapted to be released when the plunger flange on the plunger arm engages with the ears and spreads them apart.

There may be further included a guide arrangement mounted onto the syringe barrel which guide arrangement travels in a guide track within the sleeve assembly.

Preferably, the means to move the syringe barrel with the sleeve is a resilient means such as a spring acting between the sleeve and barrel.

There may be provided a detent on the guide arrangement which engages with a recess in the sleeve assembly and thereby retains the guide arrangement and hence the syringe within the sleeve arrangement when the syringe needle has been withdrawn into the sleeve arrangement.

In a preferred embodiment the guide arrangement is a single plastic moulding which is slid over the syringe barrel and fastened in place by adhesive or by an interference fit. Alternatively the guide made be a two component piece which is clamped round the syringe and fastened together in the damped position such as by ultrasonic welding or by adhesive or by thermal welding.

In a preferred embodiment of the invention the sleeve tube may be comprised of two separate components so that the syringe can be placed between the two components and then the components joined together to make the sleeve. Preferably once the two components of this sleeve are joined together they cannot be disassembled.

The two components may join longitudinally so that there are two sleeve halves joined longitudinally or they may join laterally so that there are two sleeve parts joined laterally.

There may be further provided a cap on the end of the sleeve tube to cover the needle when it is extending from the sleeve tube.

The sleeve arrangement may terminate at the aperture end of the sleeve in a finger arrangement so that the a cap on the syringe may be pushed through the finger arrangement when assembling the sleeve assembly and syringe arrangement.

### BRIEF DESCRIPTION OF THE DRAWINGS

This then generally describes the invention but to assist with understanding reference will now be made to the accompanying drawings which show preferred embodiments of the invention.

In the drawings

FIG. 1 shows a first embodiment of a combination syringe and sleeve assembly in perspective view;

FIG. 2 shows an embodiment of syringe assembly suitable for use with the present invention;

FIG. 3 shows a first stage of assembly of the upper sleeve component onto the syringe assembly;

FIG. 4 shows a next stage of assembly with the guide arrangement ready to be mounted onto the syringe barrel;

FIG. 5 shows a next stage of assembly with the guide arrangement mounted onto the syringe barrel;

FIG. 6 shows a next stage of assembly with spring mounted onto the syringe barrel;

FIG. 7 shows a longitudinal section of the syringe assembly in the sleeve assembly in the as supplied condition;

FIG. 8 shows a further longitudinal section of the syringe assembly in the sleeve assembly but with the plunger of the syringe in the position as at the completion of injecting;

FIG. 9 shows a longitudinal section of the syringe assembly in the sleeve assembly with the syringe retracted;

FIG. 10 shows a further longitudinal section of the syringe assembly in the sleeve assembly with the syringe retracted;

FIG. 11 shows an alternative embodiment of a combination of a syringe and sleeve assembly in perspective view;

FIG. 12 shows the embodiment FIG. 11 in a perspective view from the other end; and

FIG. 13 shows an exploded perspective view of the invention according to the embodiment of FIGS. 11 and 12.

### DESCRIPTION OF PREFERRED EMBODIMENT

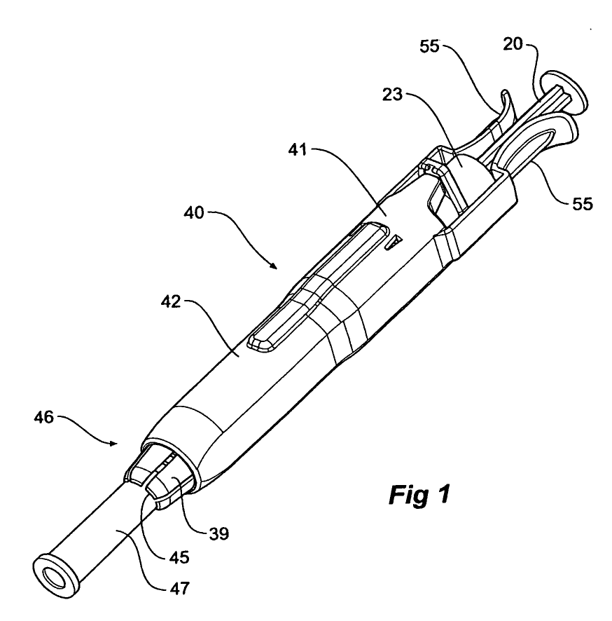

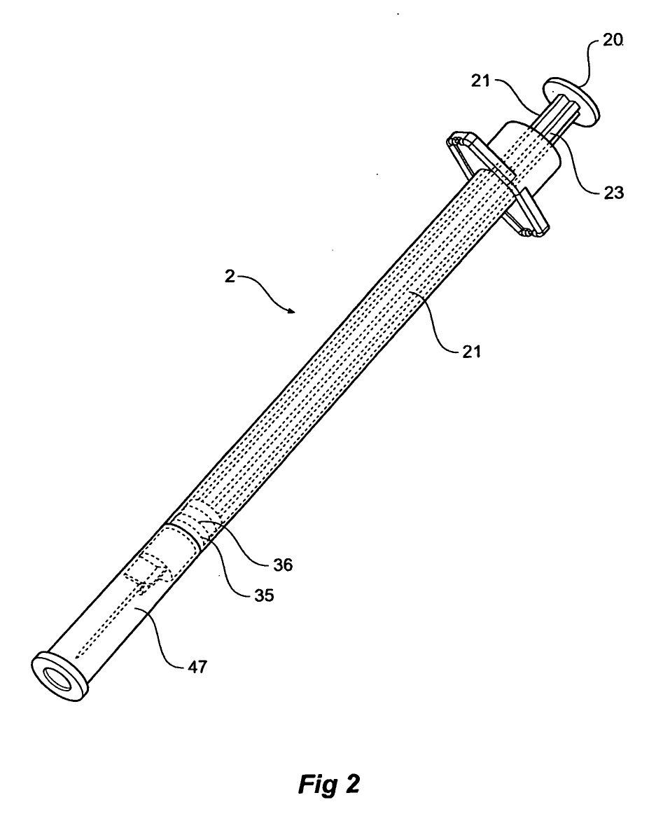

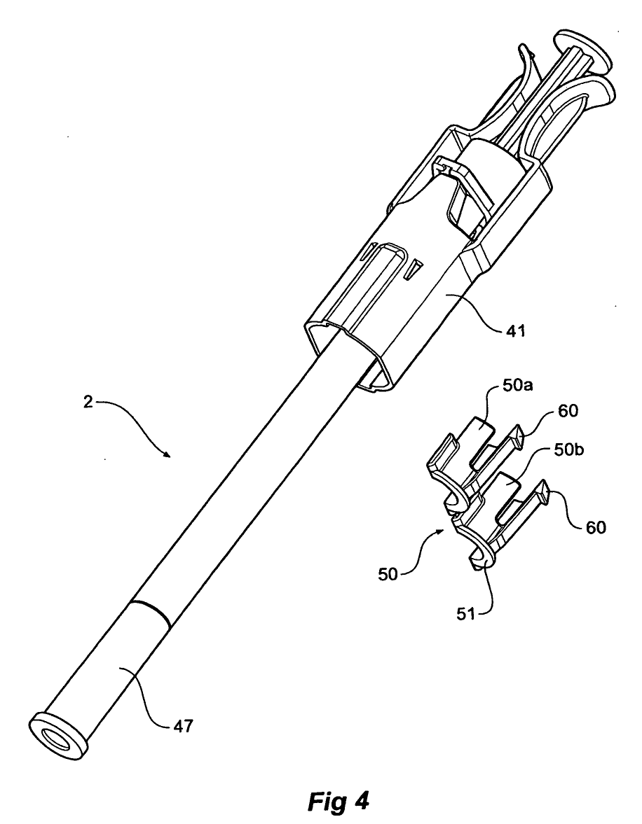

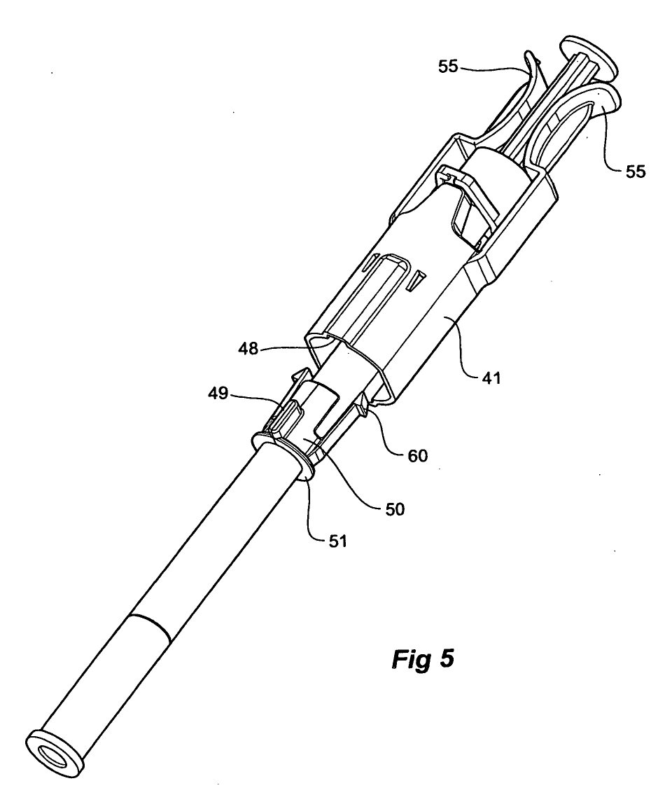

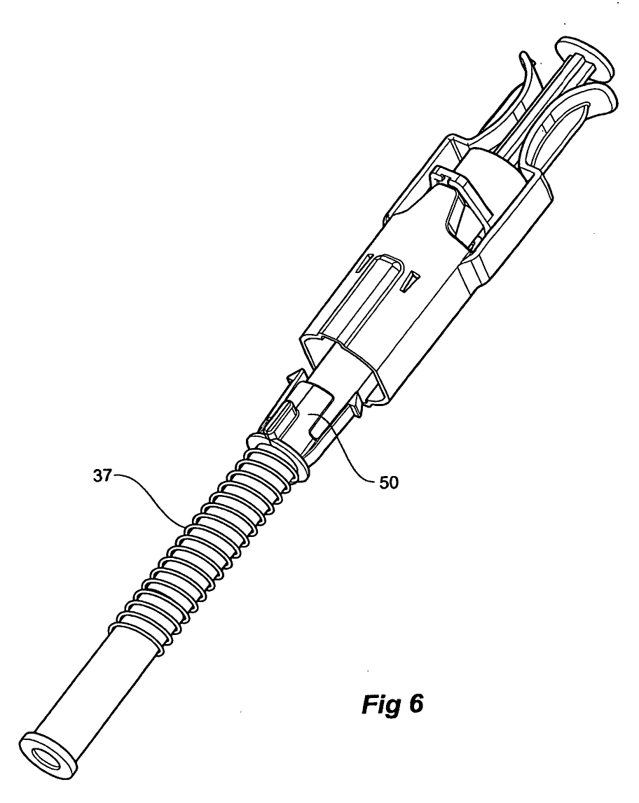

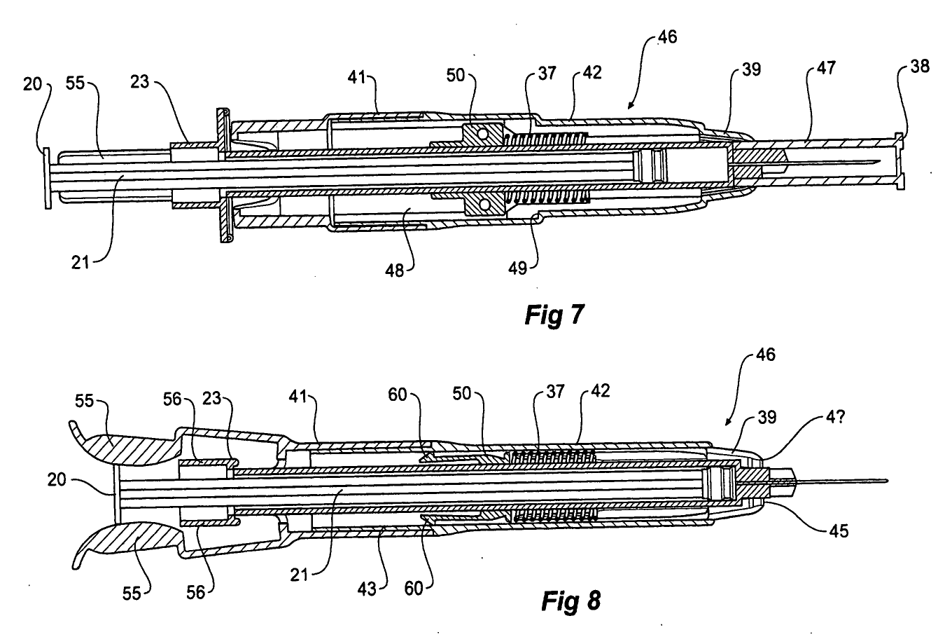

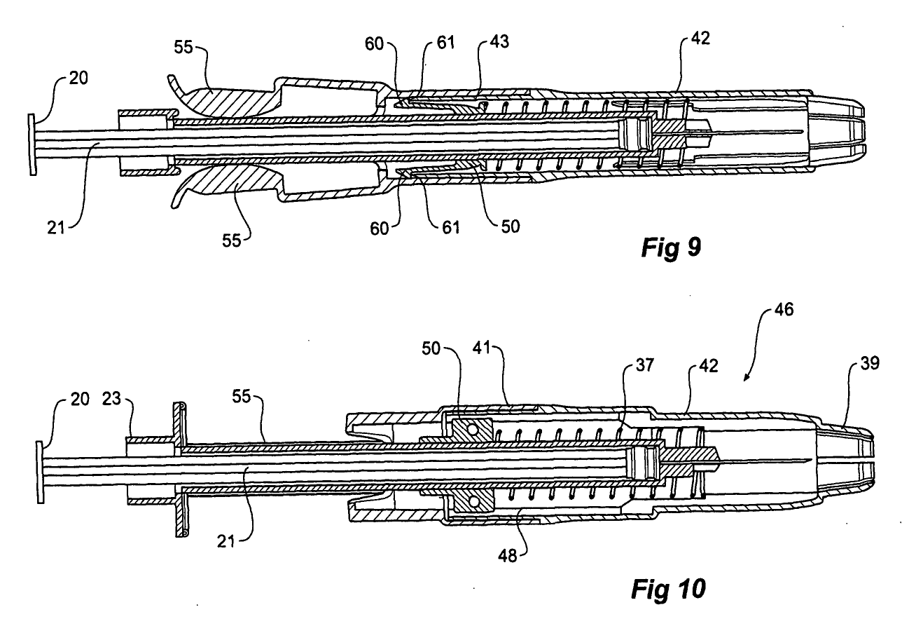

Now looking more closely at the drawings and in particular the embodiment shown in FIGS. 1 to 10. FIG. 1 shows the assembled device and FIGS. 2 to 6 show various stages of assembly. FIGS. 7 to 10 show cross sectional views at various stages of operation.

In this embodiment the sleeve assembly 40 comprises an upper portion 41 and a lower portion 42. The two sections joined together by the plug portion 43 on the lower portion engaging within the upper portion 41 and the detents 44 engaging with shoulders (not shown) within the upper portion 41 to permanently retain the two portions together once assembled. The lower portion 42 has at its end 46 an aperture 45 through which the hypodermic needle of a syringe passes in use. Around the aperture 45 at the end 46 are a number of fingers 39 extending longitudinally. These fingers 39 are slightly resilient and as the syringe 2 cap 47 is pushed through the lower portion during assembly the fingers spread slightly to allow the larger portion 38 of the cap 47 to pass through the aperture 45.

The cap 47 remains fitted onto the end of the syringe 2 to protect the needle before use.

Within the body portion 42 is a guide track 48 in which the edges 49 of a guide assembly 50 travel.

The guide assembly 50 is made up from two identical guide portions 50a and 50b clamped around a syringe body and fastened together by ultrasonic welding or other technique.

A spring 37 is placed within the lower sleeve portion 42 so that one end engages against an abutment inside of the end 46 of the sleeve tube and the other end engages against the end 51 of the guide assembly.

The upper portion 41 of the sleeve assembly has ears 55 and a retaining catch or shoulder 56 which in use engages against the upper portion 23 of a syringe to hold it in place.

When the syringe is used and the plunger flange 20 is fully pushed into the syringe the plunger flange 20 spreads apart the ears 55 by engaging on the tapered surfaces 58 which releases the upper portion 23 of the syringe 2 from the retaining catch or shoulder 56 which under the resilient action of the spring 37 withdraws the entire syringe and hence the needle into the sleeve assembly 40. At the same time the detent 60 on the guide assembly 50 hooks over and engages with an abutment 61 on the socket portion 43 of the lower sleeve portion 42 and prevents the syringe from being pushed out of the sleeve assembly again.

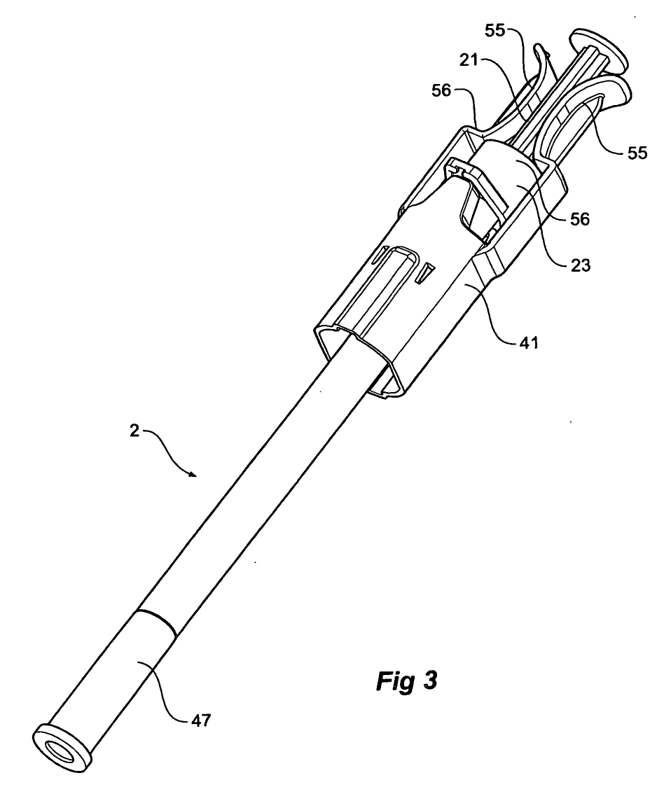

The stages of assembly of the syringe are shown in FIGS. 3 to 6.

As shown in FIG. 3 the syringe 2 is pushed into the upper sleeve portion 41 from the ear 55 end until the upper portion 23 of the syringe engages under the shoulders 56 on the ears 55. The plunger flange 20 on the plunger arm 21 remains engages the ears 55 but does not spread them. Hence the plunger piston 36 is moved slightly from the end 35 of the syringe 2.

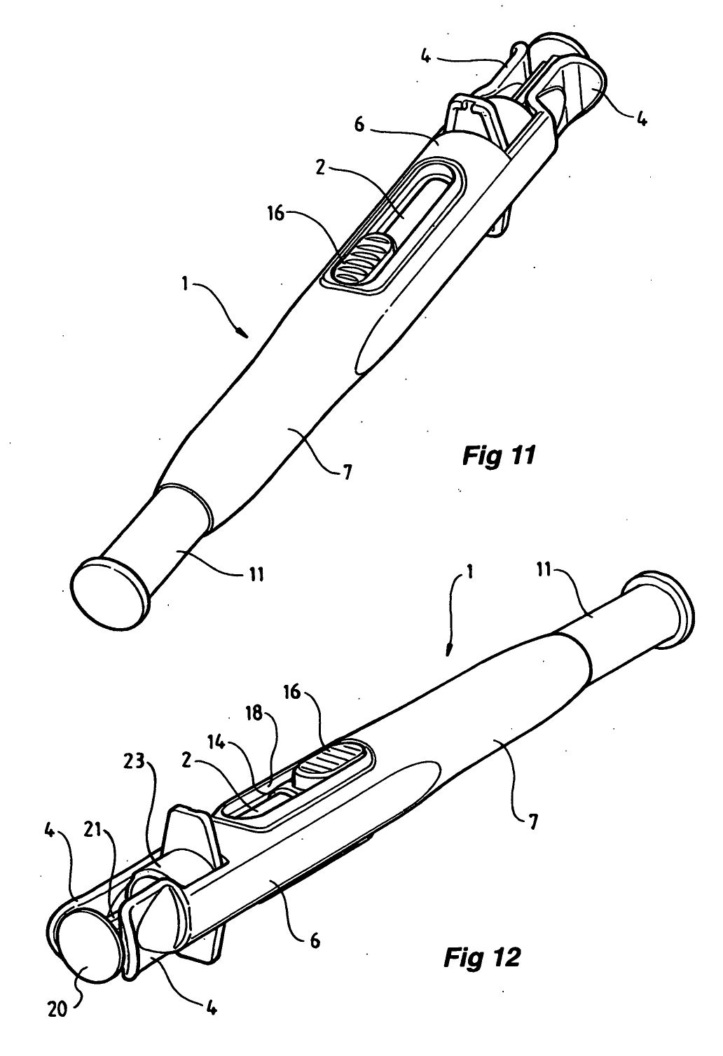

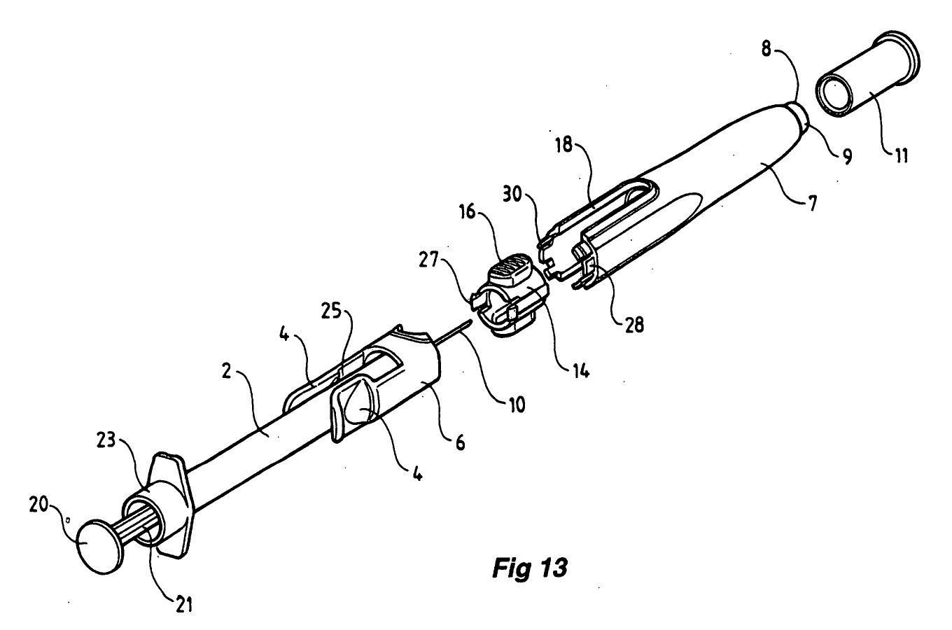

An alternative embodiment of syringe and sleeve assembly is shown in FIGS. 11 to 13.

In this embodiment it will be seen that the sleeve generally shown as 1 surrounds the syringe 2. The sleeve 1 has ears 4 on a upper sleeve portion 6 which is joined to a lower sleeve portion 7. The lower sleeve portion 7 has an aperture 8 at its end 9 through which hypodermic needle 10 on the syringe 2 extends. The needle before use is covered by a cap 11.

A guide 14 is mounted to the syringe tube so that when the needle is extending through the aperture 8 the guide 14 has its finger gripping portions 16 positioned in the elongate slot 18 in the side of the sleeve portion 7 at the lower end as shown in FIGS. 1 and 2.

As the syringe is being used the plunger flange 20 on the plunger arm 21 is pushed fully in and at that position it engages with the inside portions of the curved out portions of the ears 4 which will spread them apart so that the end 23 of the syringe tube 2 is released from the hook portion 25 of the ears and then the thumb grip 16 on the guide 14 can be slid back along the elongate slot 18 to withdraw the needle 10 into the sleeve assembly 1.

At this stage the detents 27 on the guide 14 engage against a shoulder 28 inside the sleeve 1 and this prevents the needle from being exposed again. Resilient means such as a spring within the sleeve (not shown) may be provided to move the syringe body and hence the needle back inside the sleeve assembly so that the detent portions 27 engage the shoulder 28.

The lower portion of the body 7 and the upper portion of the body 6 are joined together and the catches 30 extending from the lower portion engage in the upper portion to permanently hold the two components together once assembled.

To assemble the sleeve and syringe assembly the upper sleeve portion 6 is slid onto the syringe until the end of the syringe end 23 engages against the retaining hooks 25 on the ears 4. The guide 14 is then slid onto the needle body and secured in place and then the lower sleeve portion 7 is mounted onto the upper sleeve portion 6.

By this arrangement a safety syringe is provided because automatically when the plunger is pushed fully downwards the syringe is released and resilient pressure withdraws the needle into the sleeve body and the detent engaging on the shoulder prevents the syringe from being used again.

Throughout this specification various indications have been given as to the scope of the invention but the invention is not limited to any one of these but may reside in two or more of these combined together. The examples are given for illustration only and not for limitation.

1. A syringe retraction arrangement adapted to automatically retract a syringe into a container upon full depression of a plunger of the syringe into the syringe thereby avoiding the potential for needle stick injuries.

2. A syringe retraction arrangement as in claim 1 wherein the syringe is resiliently biased to retract into the container and has a retaining arrangement to prevent it retracting and has release means for the retaining arrangement which are engaged upon complete depression of the plunger to operate the release means.

3. A syringe retraction arrangement including a sleeve assembly and syringe, the syringe being of a type having a syringe barrel with a plunger bulb and a plunger arm terminating in a plunger flange, the plunger adapted to move in the syringe barrel and a hypodermic needle extending from the syringe barrel, the sleeve assembly including a sleeve to receive the syringe with an aperture at one end of the sleeve through which the hypodermic needle extends in use, a pair of ears on the sleeve on the end remote from the aperture, the ears adapted to engage the end of the syringe barrel remote from the hypodermic needle and to release the syringe barrel by disengagement with the end of the barrel when the ears are spread apart and means to move the syringe barrel within the sleeve assembly so as to move the syringe to withdraw the hypodermic needle into the sleeve tube when the end of the syringe barrel is released from the ears.

4. A syringe retraction arrangement as in claim 3 wherein the end of the syringe barrel is adapted to be released when the plunger flange on the plunger arm engages with the ears and spreads them apart.

5. A syringe retraction arrangement as in claim 3 further including a guide arrangement mounted onto the syringe barrel which guide arrangement travels in a guide track within the sleeve assembly.

6. A syringe retraction arrangement as in claim 3 wherein the means to move the syringe barrel within the sleeve is a resilient means.

7. A syringe retraction arrangement as in claim 3 wherein the means to move the syringe barrel within the sleeve is a spring acting between the sleeve assembly and the barrel.

8. A syringe retraction arrangement as in claim 3 further including a detent on the guide arrangement which engages with a recess in the sleeve assembly and thereby retains the guide arrangement and hence the syringe within the sleeve assembly when the syringe needle has been retracted into the sleeve assembly.

9. A syringe retraction arrangement as in claim 3 wherein the guide arrangement is a single plastic molding which is slid over the syringe barrel and fastened in place by adhesive or by an interference fit.

10. A syringe retraction arrangement as in claim 3 wherein the guide arrangement is a two component piece which is clamped round the syringe barrel and fastened together in the clamped position such as by ultrasonic welding or by adhesive or by thermal welding.

11. A syringe retraction arrangement as in claim 3 wherein the sleeve assembly is comprised of two separate components so that the syringe can be placed between the two components and then the components joined together to make the sleeve assembly.

12. A syringe retraction arrangement as in claim 11 wherein the two components of the sleeve assembly are adapted to be joined together so that they cannot be disassembled.

13. A syringe retraction arrangement as in claim 11 wherein the two components are joined longitudinally so that there are two sleeve parts joined longitudinally.

14. A syringe retraction arrangement as in claim 11 wherein the two components are joined laterally so that there are two sleeve parts joined laterally.

15. A syringe retraction arrangement as in claim 11 further including a cap on the end of the sleeve assembly to cover the needle when it is extending from the sleeve assembly.

16. A syringe retraction arrangement as in claim 11 wherein the sleeve assembly terminates at the aperture end of the sleeve in a finger arrangement so that a cap on the syringe may be pushed through the finger arrangement when assembling the sleeve assembly and syringe.

17. A syringe retraction arrangement as in claim 4 further including a guide arrangement mounted onto the syringe barrel which guide arrangement travels in a guide track within the sleeve assembly.
